(19) United States
(12) Patent Application Publication
(10) Pub. No.: US 2024/0290420 A1
(43) Pub. Date: Aug. 29, 2024

(54) SYSTEMS AND METHODS FOR COMPUTATIONAL DRUG DISCOVERY

(71) Applicant: TandemAI Suzhou Co., Ltd., Suzhou (CN)

(72) Inventors: Albert Pan, New York, NY (US); Frederick Parsons, Montreal (CA); Paul Pillot, Montreal (CA)

(73) Assignee: TandemAI Suzhou Co., Ltd., Suzhou (CN)

(21) Appl. No.: 18/387,583

(22) Filed: Nov. 7, 2023

Related U.S. Application Data
(60) Provisional application No. 63/448,387, filed on Feb. 27, 2023.

(51) Int. Cl. G16B 15/30

(52) U.S. Cl. CPC G16B 15/30

(57) ABSTRACT

Systems, methods, and instrumentalities are described for computational drug discovery. Computational experiments may be created and performed based on a ligand dataset and a receptor against which the ligands are evaluated. The computational experiments may include pose and free energy calculations, from which mapping and alchemical properties of the ligands may be determined. Various forms of feedback regarding the status, progress, and results of the computational experiments may be provided, and a user may add, modify or re-run an experiment based on the feedback to further inspect the affinity and/or selectivity of the ligands.

### CROSS-REFERENCE TO RELATED APPLICATIONS

This application claims the benefit of Provisional U.S. Patent Application No. 63/448,387, filed Feb. 27, 2023, the disclosure of which is incorporated herein by reference in its entirety.

### BACKGROUND

Conventional drug discovery processes rely almost exclusively on developing and running assays and performing chemical synthesis in “wet” laboratories. With the advancement of computer technologies, computation based drug discovery techniques have been increasingly employed in recent years to create “dry” environments in which high throughput screening, compound optimization, and/or binding affinity prediction, etc. may be performed based on calculation and/or simulation. Despite these developments, however, turnkey computational drug discovery solutions incorporating project management, molecular simulation, and data analysis features are still lacking.

### SUMMARY

Disclosed herein are systems, methods, and instrumentalities associated with computational drug discovery. In accordance with embodiments of the disclosure, a computational drug discovery system or platform may obtain a dataset, which may include information regarding a plurality of ligands. The computational drug discovery system may determine a computational experiment to be conducted using the dataset. The computational experiment may include a free energy calculation and a pose calculation, wherein the free energy calculation may be designed to determine respective binding free energies between a receptor and the plurality of ligands, and wherein the pose calculation may be designed to determine the respective manners in which the plurality of ligands binds with the receptor that have resulted in the binding free energies determined by the free energy calculation. The computational drug discovery system may conduct the computational experiment by performing at least the free energy calculation and the pose calculation, and provide feedback regarding the computational experiment. The feedback may include a three-dimensional (3D) representation of the respective manners in which one or more of the plurality of ligands bind with the receptor. The feedback may further include an indication of the respective binding free energies between the receptor and the one or more ligands.

In examples, the dataset and the receptor associated with the computational experiment may be received via a user input. In examples, after initiating an original run of the computational experiment, the computational drug discovery system may receive an indication of a change to the pose calculation or the free energy calculation (e.g., a change to one or more parameters of the pose calculation or the free energy calculation). In response, the computational drug discovery system may initiate an additional run of the computational experiment based on the indicated change while maintaining the original run of the computational experiment. The computational drug discovery system may provide a graphical representation of the status of the computational experiment, wherein the graphical representation may depict the original run and the additional run of the computational experiment as two forked branches.

In examples, the free energy calculation described herein may include a relative binding free energy (RBFE) calculation associated with at least a subset of the plurality of ligands, and the feedback described herein may further include a graphical representation of the subset of the plurality of ligands. Such a graphical representation may include nodes and edges, wherein each node may represent a respective ligand and each edge may represent a respective RBFE associated with two ligands connected by the edge. In examples, in response to receiving a user input that indicates a selection of an edge in the graphical representation, the computational drug discovery system may provide a 3D representation of the two ligands connected by the selected edge, wherein the 3D representation may indicate corresponding atoms of the two ligands that are mapped during the RBFE calculation. In examples, in response to determining that corresponding atoms of multiple ligands are selected by a user, the computational drug discovery system may establish a mapping between the corresponding atoms and perform the RBFE calculation based on the established mapping.

In examples, as part of the computational experiment, the computational drug discovery system may simulate respective interactions between the receptor and each of the plurality of ligands, and the feedback described herein may further include an indication of the respective interactions between the receptor and each of the plurality of ligands. For example, the feedback may include a comparison of the pattern in which the interactions occur between the receptor and a first ligand and the pattern in which the interactions occur between the receptor and a second ligand. The feedback may also include a heat map that compares the respective patterns in which interactions occur between the receptor and the plurality of ligands. In examples, the feedback may also include a molecular dynamics (MD) trajectory movie that depicts the interactions between the receptor and the ligands. Such a trajectory movie may include multiple frames, wherein each frame may correspond to a step in the simulation and depict the interactions between the receptor and the ligands during the step.

### BRIEF DESCRIPTION OF THE DRAWINGS

FIG. 1 is a simplified block diagram illustrating an example of a computational drug discovery system.

FIGS. 2A-2O illustrate example user interfaces associated with creating, executing, monitoring, and/or managing computational experiments.

FIG. 3 illustrates an example of feedback associated with a pose calculation.

FIG. 4 illustrates an example of feedback associated with a force calculation.

FIG. 5 illustrates an example of feedback associated with network mapping.

FIGS. 6A-6C illustrate examples of feedback associated with a free energy calculation.

FIGS. 7A-7D illustrate examples of analytics that may be determined based on a free energy calculation.

FIG. 8 illustrates an example process for computational drug discovery.

FIG. 9 illustrates an example of a computing device that may be used to accomplish one or more of the computational drug discovery tasks described herein.

### DETAILED DESCRIPTION

A more detailed understanding may be had from the following disclosure, given by way of example in conjunction with the accompanying drawings. The disclosure includes a variety of aspects, including tools, features, examples, models, approaches, etc. Many of these aspects are described with specificity and, at least to show the individual characteristics, are often described in a manner that may sound limiting. However, this is for purposes of clarity in description, and may not limit the application or scope of those aspects. Indeed, all of the different aspects may be combined and interchanged to provide further aspects. Moreover, the aspects may be combined and interchanged with aspects described in earlier filings as well.

The aspects described and contemplated in this disclosure may be implemented in many different forms. The figures provided herein may provide some examples, but other examples are contemplated. The discussion of the figures may not limit the breadth of the implementations. These and other aspects may be implemented as a method, an apparatus, and/or a computer readable medium (e.g., storage medium) comprising (e.g., having stored thereon) instructions for carrying out any of the methods described.

Various methods are described herein, and each of the methods includes one or more steps or actions for achieving the described method. Unless a specific order of steps or actions is required for proper operation of the method, the order and/or use of specific steps and/or actions may be modified or combined. Additionally, terms such as “first”, “second”, etc. may be used in various examples to modify an element, component, step, operation, etc., such as, for example, a “first computational experiment” and a “second computational experiment.” Use of such terms may not imply an ordering to the modified operations unless specifically required. So, in this example, the first computational experiment need not be performed before the second computational experiment, and may occur, for example, before, during, or in an overlapping time period with the second computational experiment.

Various numeric values are used in examples described in the present disclosure. These and other specific values are for purposes of describing examples and the aspects described are not limited to these specific values. When a figure is presented as a flow diagram, it should be understood that it also provides a block diagram of a corresponding apparatus. Similarly, when a figure is presented as a block diagram, it should be understood that it also provides a flow diagram of a corresponding method/process.

The implementations and aspects described herein may be implemented in, for example, a method or a process, an apparatus, a software program, or a data stream. Even if only discussed in the context of a single form of implementation (for example, discussed only as a method), the implementation of features discussed may also be implemented in other forms (for example, an apparatus or program). An apparatus may be implemented in, for example, appropriate hardware, software, and firmware. The methods may be implemented in, for example, a processor, which refers to processing devices in general, including, for example, a computer, a microprocessor, an integrated circuit, or a programmable logic device. Processors also include communication devices, such as, for example, computers, cell phones, portable/personal digital assistants (“PDAs”), and other devices that facilitate communication of information between end-users.

Reference to “one example” or “an example” or “one implementation” or “an implementation”, as well as other variations thereof, means that a particular feature, structure, characteristic, and so forth described in connection with the example is included in at least one example. Thus, the appearances of the phrase “in one example” or “in an example” or “in one implementation” or “in an implementation”, as well any other variations, appearing in various places throughout this disclosure are not necessarily all referring to the same example.

Additionally, this disclosure may refer to “determining” various pieces of information. Determining the information may include one or more of, for example, estimating the information, calculating the information, predicting the information, or retrieving the information from memory. Obtaining may include receiving, retrieving, constructing, generating, and/or determining.

Further, this disclosure may refer to “accessing” various pieces of information. Accessing the information may include one or more of, for example, receiving the information, retrieving the information (for example, from memory), storing the information, moving the information, copying the information, calculating the information, determining the information, predicting the information, or estimating the information.

Additionally, this disclosure may refer to “receiving” various pieces of information. Receiving is, as with “accessing,” intended to be a broad term. Receiving the information may include one or more of, for example, accessing the information, or retrieving the information (for example, from memory). Further, “receiving” is typically involved, in one way or another, during operations such as, for example, storing the information, processing the information, transmitting the information, moving the information, copying the information, erasing the information, calculating the information, determining the information, predicting the information, or estimating the information.

It is to be appreciated that the use of any of the following “/”, “and/or”, and “at least one of”, for example, in the cases of “A/B”, “A and/or B” and “at least one of A or B”, is intended to encompass the selection of the first listed option (A) only, or the selection of the second listed option (B) only, or the selection of both options (A and B). As a further example, in the cases of “A, B, and/or C” and “at least one of A, B, or C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as is clear to one of ordinary skill in this and related arts, for as many items as are listed.

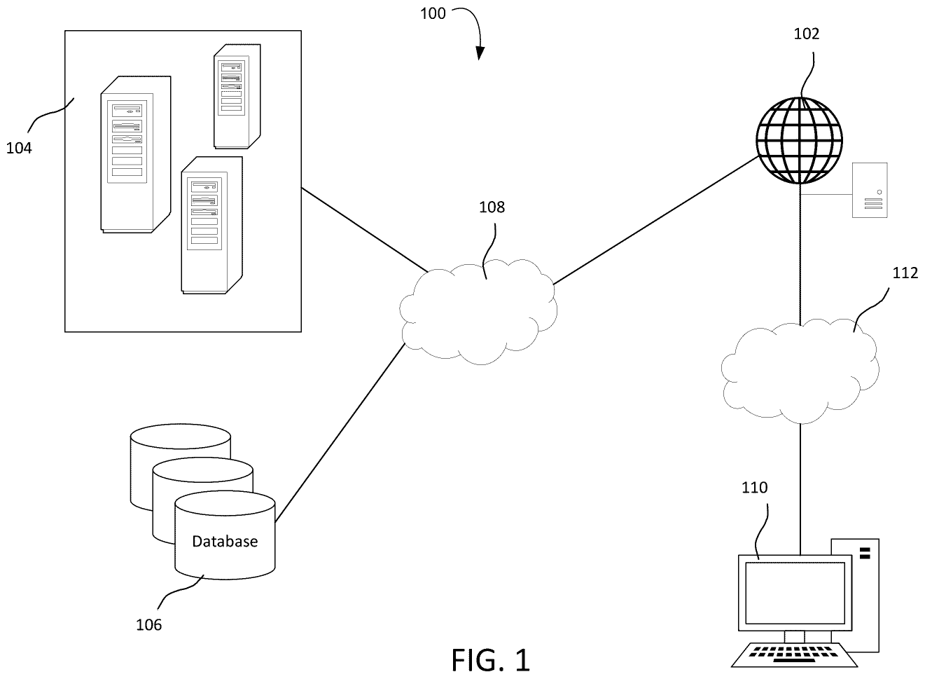

FIG. 1 illustrates an example of a computational drug discovery system 100 (also referred to herein as a computational drug discovery platform) in accordance with one or more embodiments of the present disclosure. As shown in FIG. 1, the computational drug discovery system may include one or more first computing devices 102 (e.g., one or more web servers), one or more second computing devices 104 (e.g., a cluster of servers), and/or one or more databases 106, which may be connected via a first communication network 108 (e.g., a private enterprise network) and configured to exchange information over the first communication network. The first computing devices 102 may also be configured to interact with a client device 110 (e.g., through a web browser of the client device) over a second communication network 112 (e.g., a public network such as the Internet) and receive instructions from the client device 110 (e.g., from a user of the client device 110) regarding one or more drug discovery related services. The first computing devices 102 may dissect the received instructions and determine, based on the instructions, one or more specific computational tasks (e.g., free energy perturbation (FEP) calculations, molecular dynamics (MD) simulations, predictive computations for binding affinity, etc.) initiated by the client device 110. The first computing devices 102 may pass these tasks to the second computing devices 104 for execution and may track the progress of the execution, for example, based on feedback (e.g., computation results, status reports, logs, etc.) provided (e.g., directly) by the second computing devices 104 and/or information stored by the second computing devices 104 in databases 106. The first computing devices 102 may provide ways for the client device 110 to monitor the status of the task execution and may present results of the task execution, such as, e.g., binding free energies, protein-ligand interactions, etc., to the client device 104. The results may be presented via various forms of reports and graphical representations including, for example, three-dimensional (3D) poses, graphs, charts, heatmaps, movies, etc.

The computational drug discovery system shown in FIG. 1 may accelerate lead optimization and hit-to-lead processes by predicting and optimizing ligand binding affinity and selectivity, identifying key residues involved in ligand binding and specific interactions, revealing the structure-activity relationship (SAR) of the ligand, and demonstrating the dynamic behavior of protein-ligand complexes. Compared to traditional drug discovery techniques, the computational drug discovery system shown in FIG. 1 may allow researchers to identify the most promising candidates for further synthetic efforts and explore a much more expanded chemical space. For example, by calculating the free energy difference between two ligands, the computational drug discovery system can identify which modifications are likely to improve or impair binding affinity, allowing researcher to focus their synthetic efforts on the most promising candidates.

It should be noted that the system diagram shown in FIG. 1 is just one example of how computation-based drug discovery may be performed in accordance with embodiments of the present disclosure. Other system architectures may also be used including, for example, an architecture in which the client device 110 may interact directly with the second computing devices 104 and/or the database 106 without the first computing devices 102.

In some embodiments of the present disclosure, computational experiments may be organized into projects. A project may be created by a user having certain privileges (e.g., a system administrator assigned project creation privileges) while additional users (e.g., member users) may be added to the project and given the ability to create, modify, and/or view the computational experiments (e.g., also referred to herein as runs) associated with the project. A computational experiment (e.g., a FEP run) may be created based at least on a dataset and a receptor. The dataset may include one or more ligands and each ligand in the dataset may also belong to other dataset(s) (e.g., there may be a many-to-many relationship between datasets and ligands).

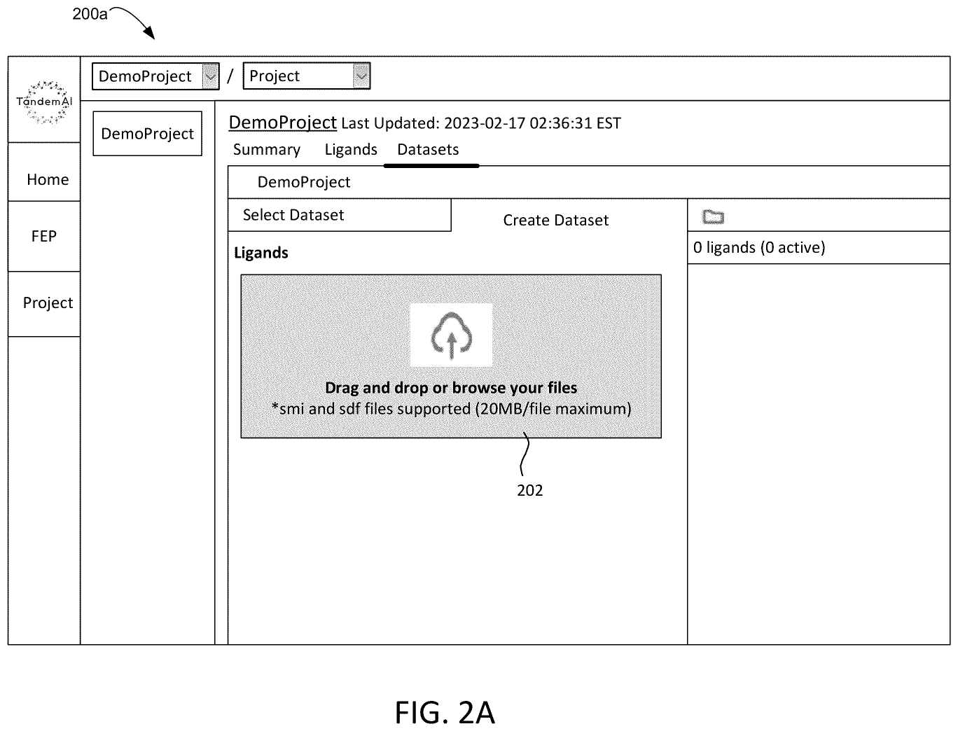

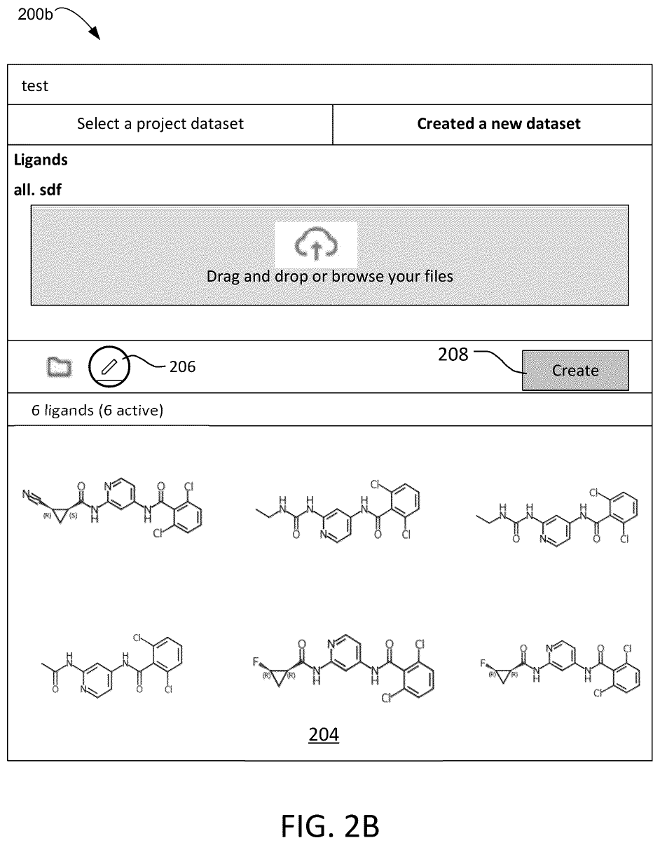

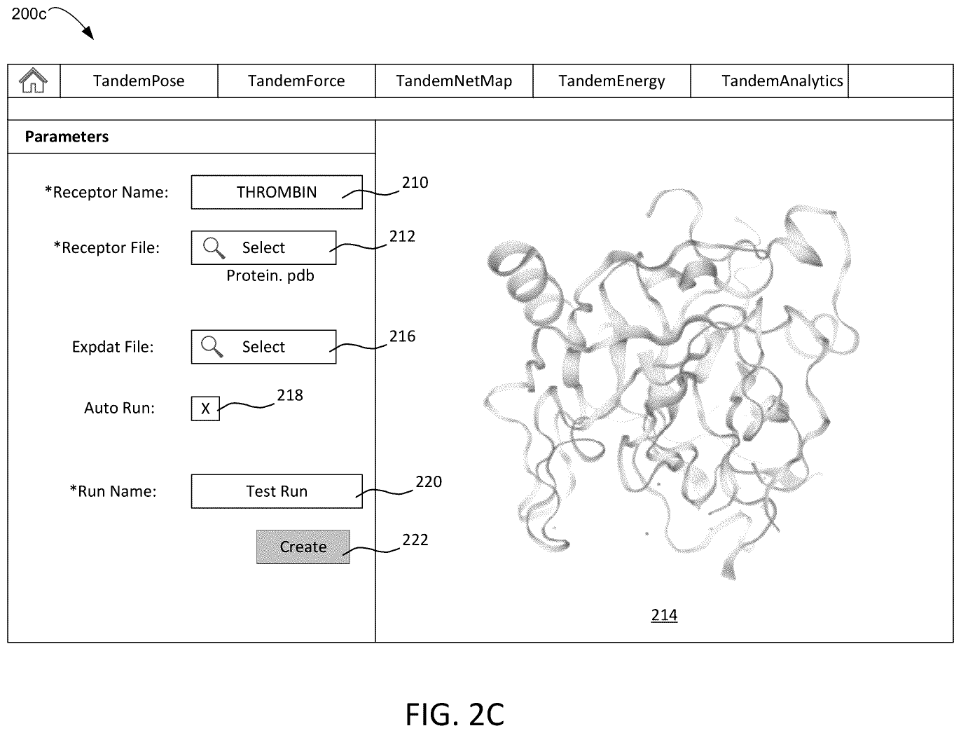

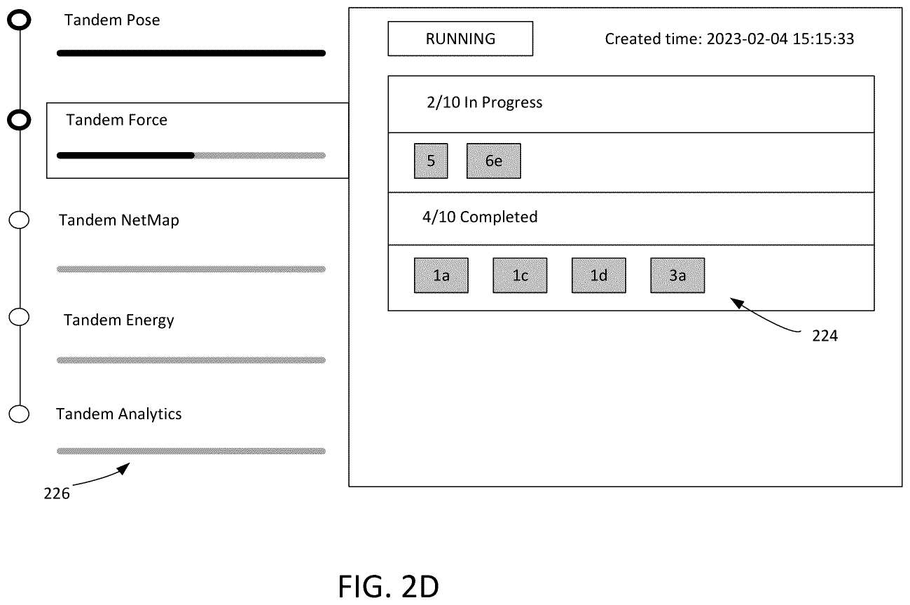

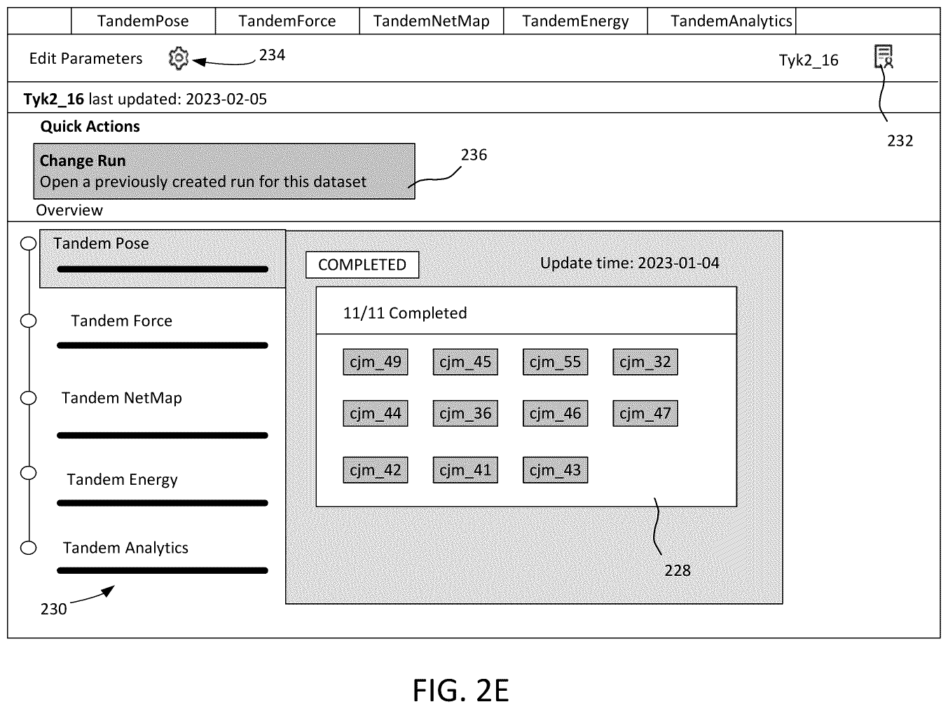

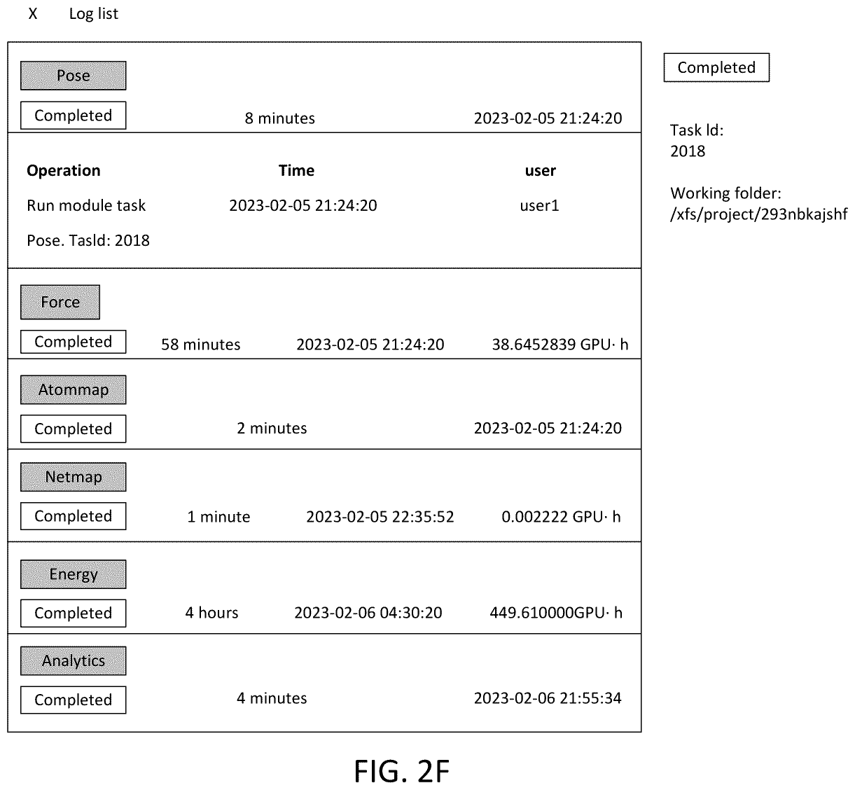

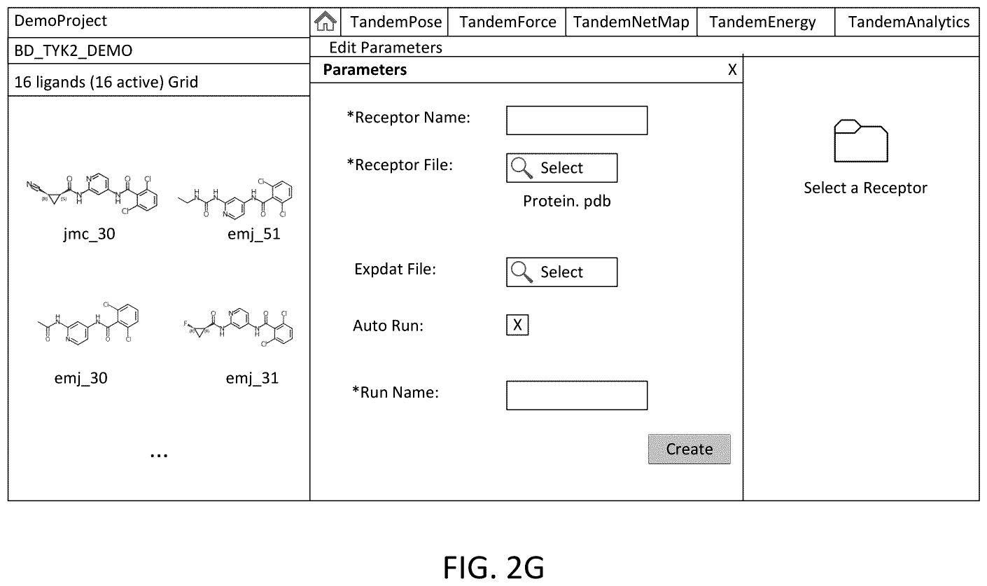

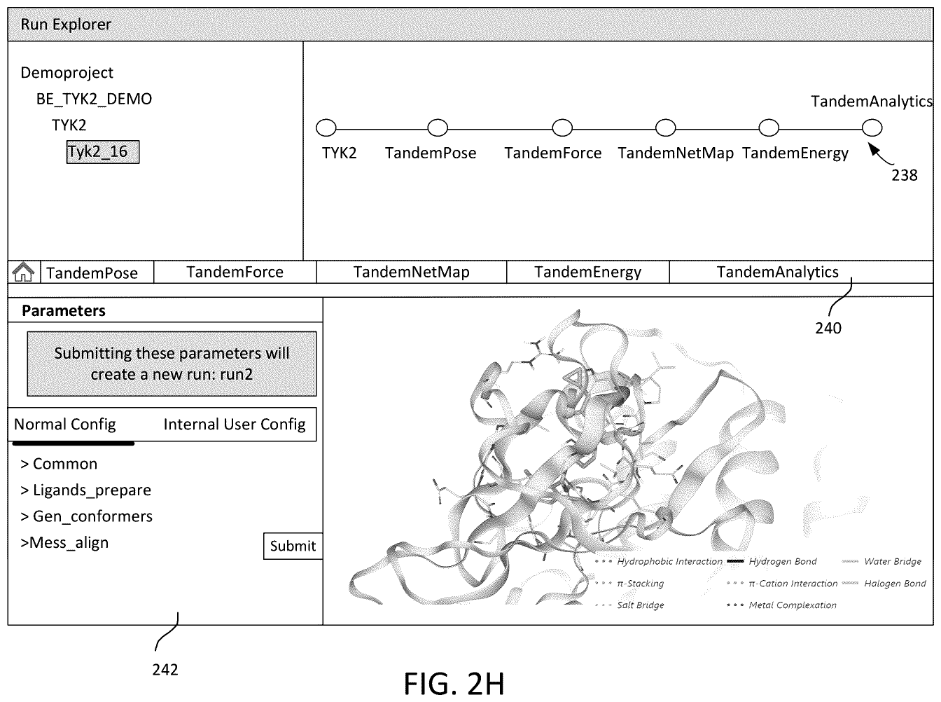

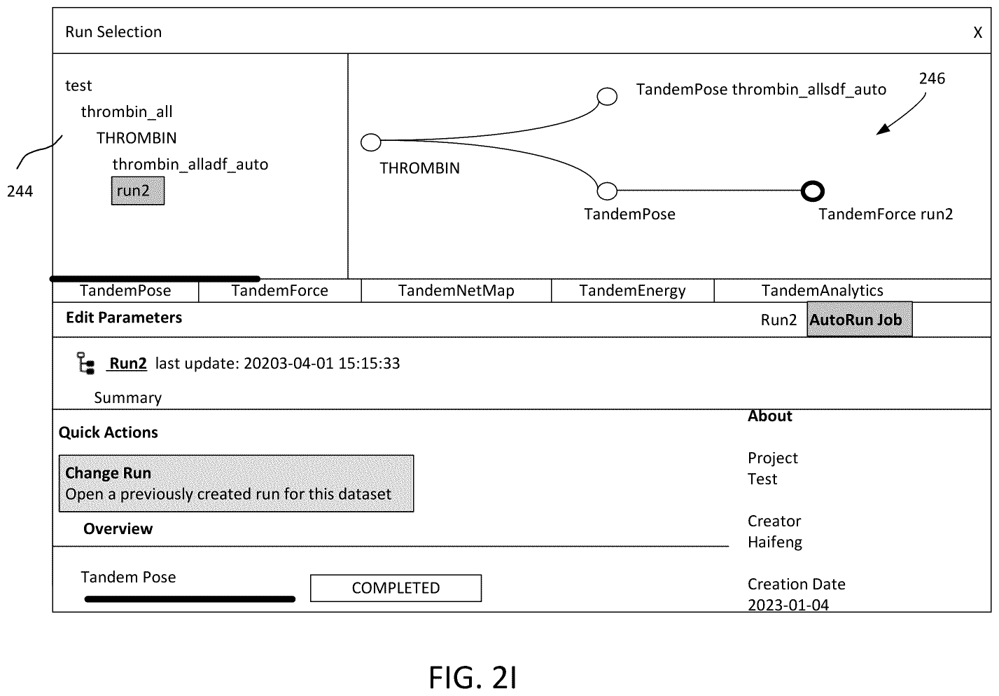

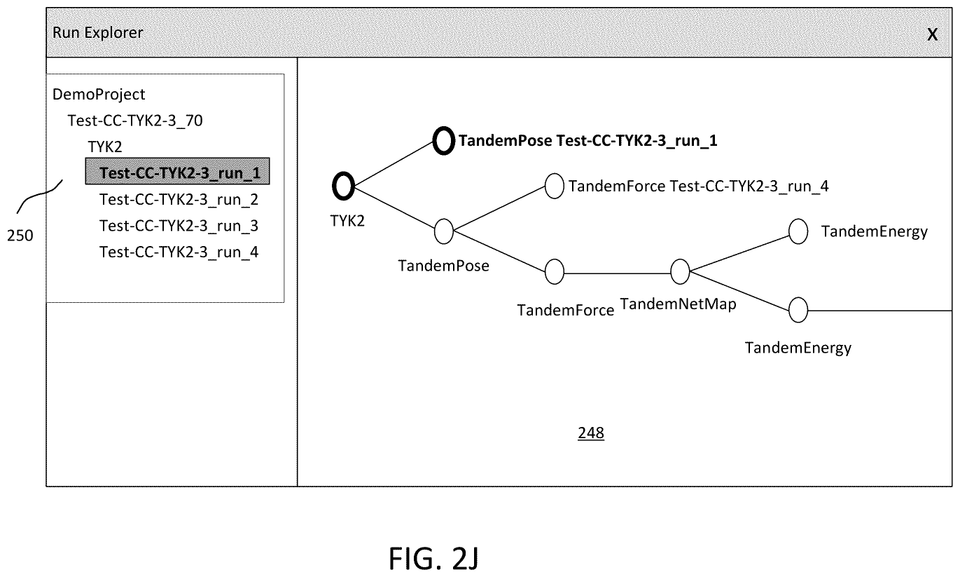

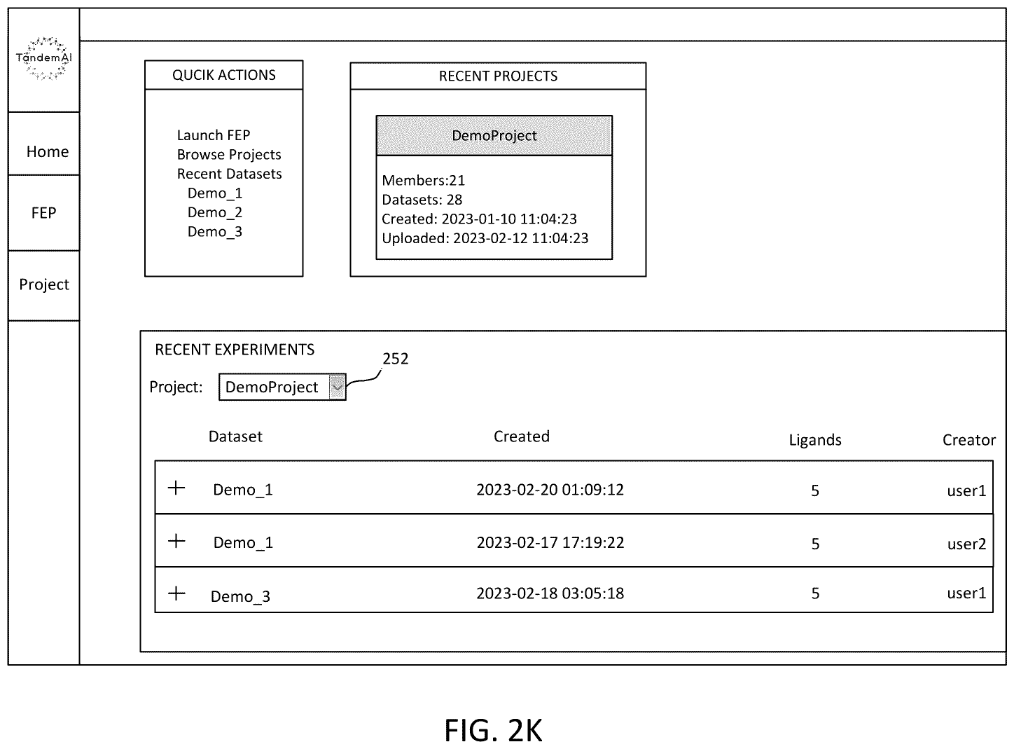

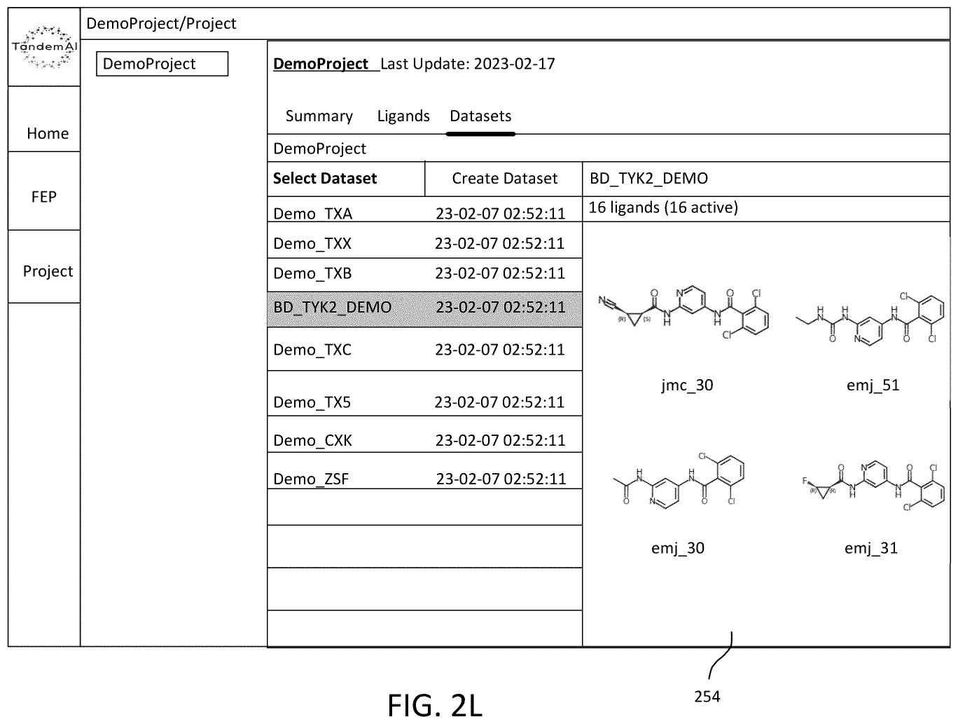

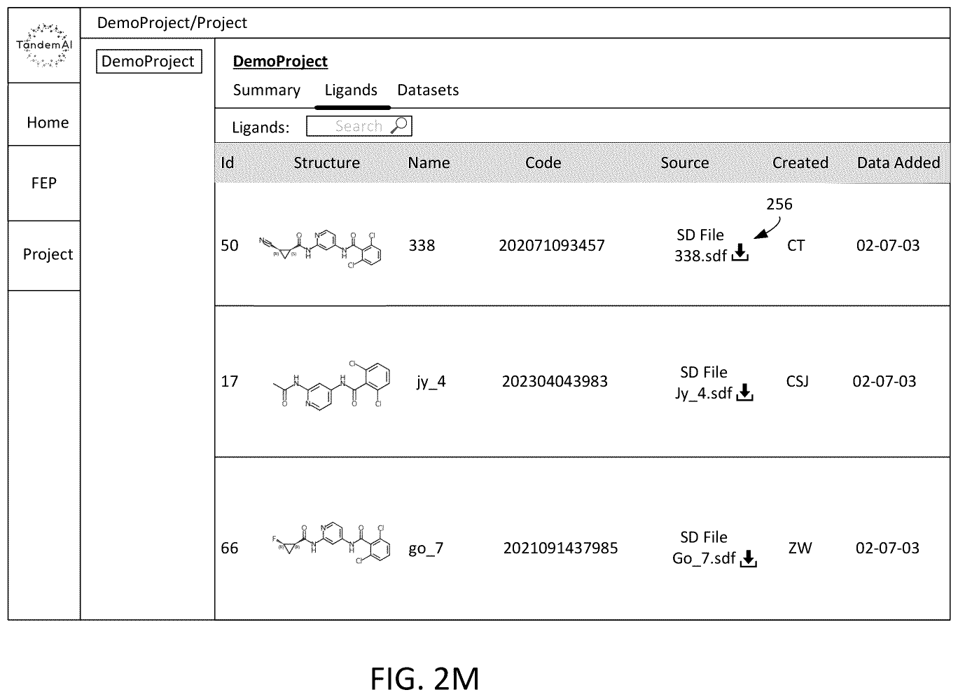

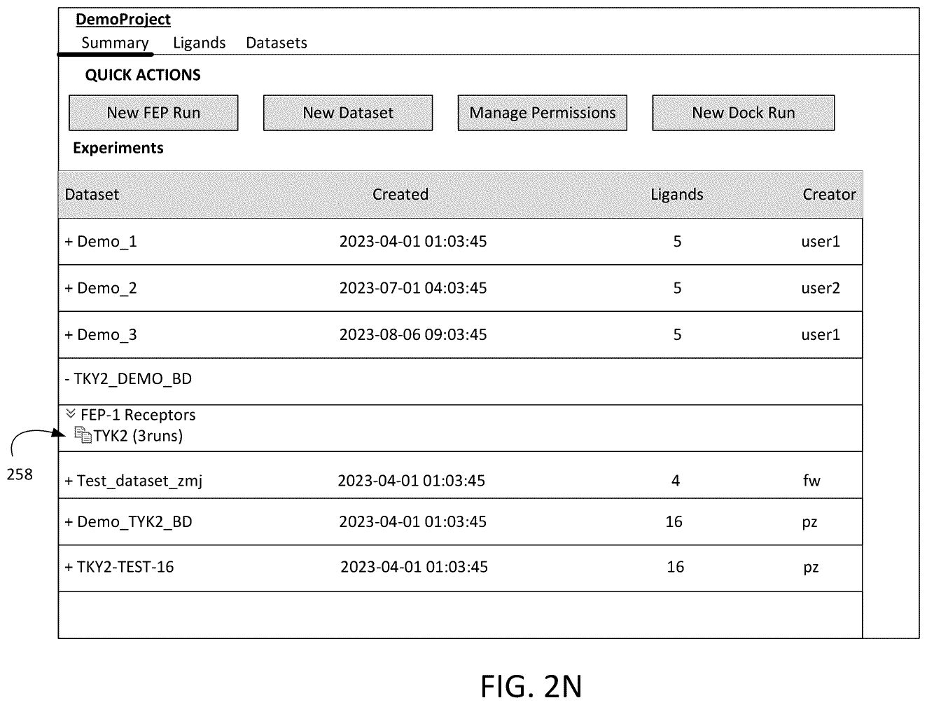

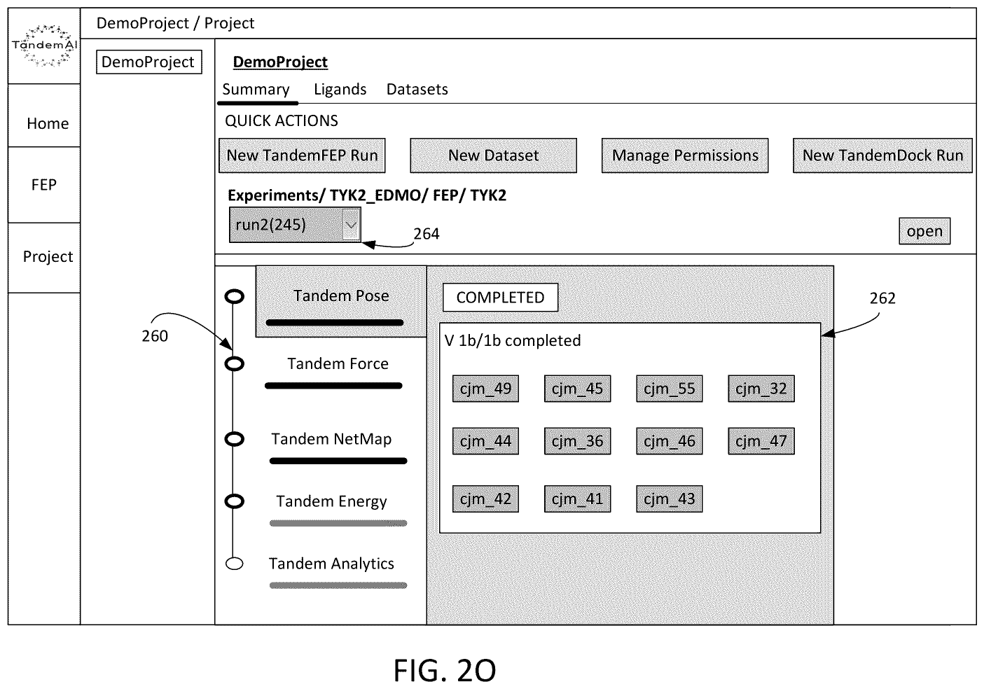

FIG. 2A illustrates an example user interface (UI) 200a that may be used to create a computational experiment according to embodiments of the present disclosure. UI 200a may be provided by the first computing device(s) 102 or the second computing device(s) 104 shown in FIG. 1 and may be displayed (e.g., in a browser) on the client device 110 shown in FIG. 1. As shown in FIG. 2A, UI 200a may be used to create a dataset within a project named “DemoProject.” The dataset may comprise one or more ligands, which may be added to the dataset using a ligand drawing tool provided by the computational drug discovery system described herein or based on a file uploaded by the user. For instance, the user may upload an .sdf or .smi file from a local or cloud-based storage area (e.g., using control component 202) and the computational drug discovery system may analyze the file content, validate the ligands contained in the file (e.g., to ensure that all structures are valid), and display the ligands including their 2D molecular structures to the user, as shown on panel 204 of the example UI 200b in FIG. 2B. Ligand information determined to be missing during the validation process may be highlighted on panel 204 and a prompt may be displayed for the user to provide the missing information. Upon completing the validation procedure, the user may create the dataset by giving it a name (e.g., “test”) or ID using control 206 and clicking the “Create” button 208, as shown in FIG. 2B.

Once the dataset has been created, the user may further create a computational experiment (e.g., a run of the computational experiment) based on the dataset. FIG. 2C illustrates an example UI 200c for accomplishing such a task. As shown in FIG. 2C, the user may use UI 200c to specify a receptor (e.g., a protein receptor) with which the ligands of the dataset may be bound. For instance, the user may provide the name of the receptor using text field 210 and/or upload a file (e.g., a protein data bank (pdb) file) that may describe the properties of the receptor (e.g., atom coordinates, disulfide bonds, helices, sheets, active sites, etc.) using control 212. Upon receiving the successfully uploaded receptor file, the computational drug discovery system described herein may display a 3D structural view of the receptor, as shown on panel 214 of FIG. 2C. In examples, UI 200c may further include a control 216 for the user to upload an experiment data file (e.g., an Expdat file) for the ligands and/or the receptor, which may include additional information about the ligands and/or the receptor obtained through laboratory experiments. UI 200c may also allow the user to specify how the present run of the computational experiment is to be performed (e.g., automatically, or manually) and/or the tasks (e.g., functional modules) to be included in the run. For example, the computational drug discovery system described herein may, by default, assign a workflow (e.g., an FEP workflow) to the computational experiment that may include a pose calculation task (e.g., illustrated as “TandemPose” in the accompanying figures), a force calculation task (e.g., illustrated as “TandemForce” in the accompanying figures), a network mapping task (e.g., illustrated as “TandemNetMap” in the accompanying figures), a free energy calculation task (e.g., illustrated as “TandemEnergy” in the accompanying figures), and an analytics collection task (e.g., illustrated as “TandemAnalytics” in the accompanying figures). The computational drug discovery system may allow the user to change the default workflow and include only a subset of the aforementioned tasks in the computational experiment.

In examples, UI 200c may include an Auto Run option 218, which, when selected, may indicate that the user desires to have the computational drug discovery system automatically perform all of the tasks (e.g., functional modules) in the present run (e.g., sequentially or in parallel). In examples, if the Auto Run option is selected by the user, the computational drug discovery system may further prompt the user to provide a reference ligand for which experimental data (e.g., experimental structural information) may be known and against which other ligands of the dataset may be docked (e.g., for relative binding free energy (RBFE) determination, for generating plausible binding modes for the other ligands, etc.). The user may choose not to use the Auto Run option and instead run one or more of the tasks described herein manually. In that case, the computational drug discovery system may allow the user to specify a reference ligand for the specific task(s) selected by the user.

The user may distinguish a new run of a computational experiment from other runs by giving the run a unique name (e.g., using text field 220 shown in FIG. 2C) and may start the new run by clicking the “Create” button 222 shown in FIG. 2C. This may trigger the computational drug discovery system (e.g., a computation engine of the system hosted on second computing device(s) 104 of FIG. 1) to conduct the computational experiment by performing the tasks associated with the run (e.g., all or a subset of the tasks described herein). For example, the computational drug discovery system may perform the free energy calculation task described herein by determining respective binding free energies between a receptor and a plurality of ligands associated with the run, and perform the pose calculation task described herein by determining the respective manners in which the ligands may bind with the receptor that result in the binding free energies determined by the free energy calculation task. The manners of ligand-receptor binding may include respective positions and/or orientations of the ligands relative to the receptor at corresponding binding sites or binding pockets. The binding free energies described herein may include absolute binding free energies (ABFE) or relative binding free energies (RBFE) that may indicate respective affinities of the ligands with the receptor in absolute or relative terms (e.g., RBFE may indicate a difference between the binding free energies of two compounds). The binding free energies and/or binding positions between the ligands and the receptor may be determined using various techniques. For example, the binding positions between the ligands and the receptor may be determined using a molecular docking technique and the corresponding relative binding free energies may then be determined by calculating the free energy changes associated with alchemically transforming one ligand to another using molecular dynamics simulation and free energy perturbation theory, or the thermodynamic integration (TI) method. The binding free energy between a ligand and the receptor may also be determined using machine learning models, such as deep learning or regression models, that may be trained on molecular features and experimental binding data to predict binding free energies.

The computational drug discovery system may perform the force calculation task described herein (referred to as “TandemForce”) by generating force fields (e.g., small molecule force fields) for the ligands included in a dataset and comparing the fit between the potential energy dihedral scan as predicted by quantum mechanical prediction for each dihedral in the dataset. The computational drug discovery system may perform the net mapping task described herein by mapping the corresponding atoms of two or more ligands involved in the free energy calculation (e.g., RBFE calculation) and generating an alchemical network for users to visualize and edit the mapping. Further, the computational drug discovery system may perform the analytics task described herein by analyzing calculation and/or simulation results from the computational experiment and presenting the analysis in a series of data rankings and graphical representations including, for example, a dynamic, 3D representation of the manners in which one or more ligands bind with the receptor, tables/lists/charts displaying the respective binding free energies between the receptor and the one or more ligands, a trajectory movie showing the interactions between the receptor and the one or more ligands, a comparison of the patterns in which the interactions between the receptor and the one or more ligands occur, etc.

The tasks (or modules) associated with a run of a computational experiment may form a workflow for the run. The tasks (or modules) may be chained together in a coherent manner and the results of one task (or module) may be used in the next. As the tasks are being performed and/or completed, feedback may be provided (e.g., via the tabs shown in some of the accompanying figures) to indicate the current state of the workflow (e.g., status and/or results may be displayed as they become available). For example, after a run is created and started by a user, the computational drug discovery system described herein may allow the user to monitor the progress and/or results of the run. FIG. 2D shows an example of monitoring the progress of a run in accordance with embodiments of the present disclosure. As shown in FIG. 2D, while the run is in progress, the computational drug discovery system may provide a live status indicator for the run, which may indicate (e.g., on panel 224 of FIG. 2D) the ligands that have been submitted to various calculations of the run, as well as the calculations that are running, queued, or have been finished (e.g., via progress bars 226 of FIG. 2D). Once the run has been completed, the computational drug discovery system may provide an indication of the complete status, for example, as illustrated by panel 228 and progress bars 230 of FIG. 2E. In response to the user activating control 232 of FIG. 2E, the computational drug discovery system may provide logs of the run, the content of which may be as illustrated by FIG. 2F.

The computational drug discovery system may allow a user to adjust the parameters of an on-going run of a computational experiment and create another run of the computational experiment using the same dataset. For example, in response to the user indicating a desire to change the parameters of the on-going run (e.g., via control 234 of FIG. 2E), the computational drug discovery system may open a new panel (e.g., as illustrated by FIG. 2G) for the user to specify the new parameters (e.g., receptor, receptor file, Expdat file, Auto Run selection, etc.). As another example, in response to the user indicating a desire to change the on-going run (e.g., via control 236 of FIG. 2E), the computational drug discovery system may provide a graphical representation of the current state of the on-going run and allow the user to edit the task(s) associated with the on-going run. FIG. 2H illustrates an example of changing a run in response to the user activating the “Change Run” control 236 of FIG. 2E. As shown in FIG. 2H, in response to the “Change Run” control being activating, the computational drug discovery system may summarize the tasks (e.g., pose, force, mapping, energy, etc.) of the on-going run via graph 238 in which each task may be represented as a node and the relationship between two sequential tasks may be represented by an edge. The user may indicate the task to be changed, for example, by selecting the corresponding node in the graph or by activating a corresponding tab 240 in the lower panel. Upon receiving such an indication, the computational drug discovery system may display the parameters associated with the selected task (e.g., in panel 242) and allow the user to make changes to those parameters.

Once the user submits the new or updated parameters for the on-going run (e.g., through the “Edit Parameters” operation illustrated by FIG. 2G or the “Change Run” operation illustrated by FIG. 2H), and if the computational drug discovery system determines that the new or changed parameters may cause the on-going run to generate different results at one or more stages of the run (e.g., for the same dataset and receptor), the computational drug discovery system may create a new run based on the new or changed parameters and may associate the new run and the on-going run (e.g., also referred to as the original run) as two related, forked tasks or operations. The computational drug discovery system may present the newly created run and the on-going run as separate branches of a graph, as illustrated by FIG. 2I. For example, as shown in FIG. 2I, the computational drug discovery system may organize and present related runs of a computational experiment (e.g., for the same dataset and receptor) via a multi-level structure 244, in which the levels may correspond to Project->Dataset->Receptor->Run. The computational drug discovery system may also provide a graph 246 in which the receptor (e.g., “THROMBIN”) and tasks (e.g., “TandemPose,” “TandemForce,” etc.)

may be represented as nodes and the sequential relationships between nodes may be represented by edges. For instance, two runs, “thrombin_allsdf_auto” and “run2”, that are associated with a dataset “thrombin_all” and the receptor “THROMBIN” may be represented in a forking network that displays where the runs have branched (e.g., due to a new task being submitted with different parameters) that may affect the outcome of one run versus another. As illustrated by the example in FIG. 2I, graph (or forking network) 246 may indicate that the user has re-run “TandemPose” with different parameters as part of “run2” related to another run “thrombin_allsdf_auto.” FIG. 2J shows another example in which a graph (e.g., a run explorer or forking network) 248 may be provided to indicate that multiple pose, force, and energy calculations were created and started for dataset “Test-CC-TYK-3_70” and receptor “TYK2.” Using the interface shown in FIG. 2J, a user may select a run (e.g., in left pane 250) and have a corresponding branch highlighted in graph 248 to visualize where the selected run has branched from other runs.

The computational drug discovery system may allow a run to be forked at any position of a workflow and may group the runs for the same dataset and receptor in a forking network. A run may be forked, for example, in response to a user creating a new run or starting a new task (e.g., a pose or energy calculation) for an existing run in which a similar task may already be executing (e.g., if a run already has a pose task and the user launches a new pose task, this may create a new forked run). A run may also be forked based on a user cancelling a task, re-initializing a task, editing an alchemical mapping of a current run, etc.

FIGS. 2K-2O illustrate examples of tools (e.g., graphical user interfaces) that may be used to monitor and/or manage project, datasets, or computational experiments after they are created. FIG. 2K illustrates an example user interface with which a user may select a project (e.g., “DemoProject”) from a project list 252 (e.g., a dropdown box) and obtain an overview of the basic information of the project (e.g., members, creation time, etc.) as well as the number of datasets and/or ligands included in the project. FIG. 2L illustrates an example user interface with which the user may select a specific dataset (“BD_TYK2_DEMO”) and see the ligands included in that dataset, together with their two-dimensional (2D) molecular structures (e.g., as shown in panel 254). FIG. 2M illustrates an example user interface with which the user may download a source data file associated with a specific ligand, for example, by clicking control 256 for the specific ligand. FIG. 2N illustrates an example user interface with which the user may obtain a summary of the datasets/ligands included in a project and/or the computational experiments or run(s) (e.g., “TYK2 (3 runs)”) associated with a specific dataset (e.g., “TYK2_DEMO_BD”), as shown at 258e. Additionally, in response to the user selecting the run(s) associated with the specific dataset, the computational drug discovery system may provide a summary of the runs including various calculations 260 (e.g., pose, force, energy, etc.) performed for the dataset of ligands, as illustrated in FIG. 2O (e.g., panel 262 of FIG. 2O shows the ligands for which the calculations 260 have been completed). The user may switch between the runs associated with the specific dataset, for example, using a dropdown box 264 that lists all the runs.

As described herein, a computational experiment (e.g., a run) created and started by a user may be conducted using the disclosed computational drug discovery system by performing the tasks or calculations configured by a user. Various feedback may be provided regarding the results of the computational experiment during and/or after the computational experiment. For example, the feedback may include a 3D representation of the manners in which a plurality of ligands binds with a receptor, as determined by the pose calculation described herein. As another example, the feedback may include plots and/or lists that indicate the respective binding free energies of the plurality of ligands with the receptor, as determined by the energy calculation described herein. As yet another example, the feedback may include representations of the interactions (e.g., hydrophobic interactions) between the ligands and the receptor. Through the feedback, the user may predict and optimize ligand binding affinity and selectivity, identify key residues involved in ligand binding and specific interactions, explore the structure-activity relationship (SAR) of the ligands, and investigate the dynamic behavior of protein-ligand complexes.

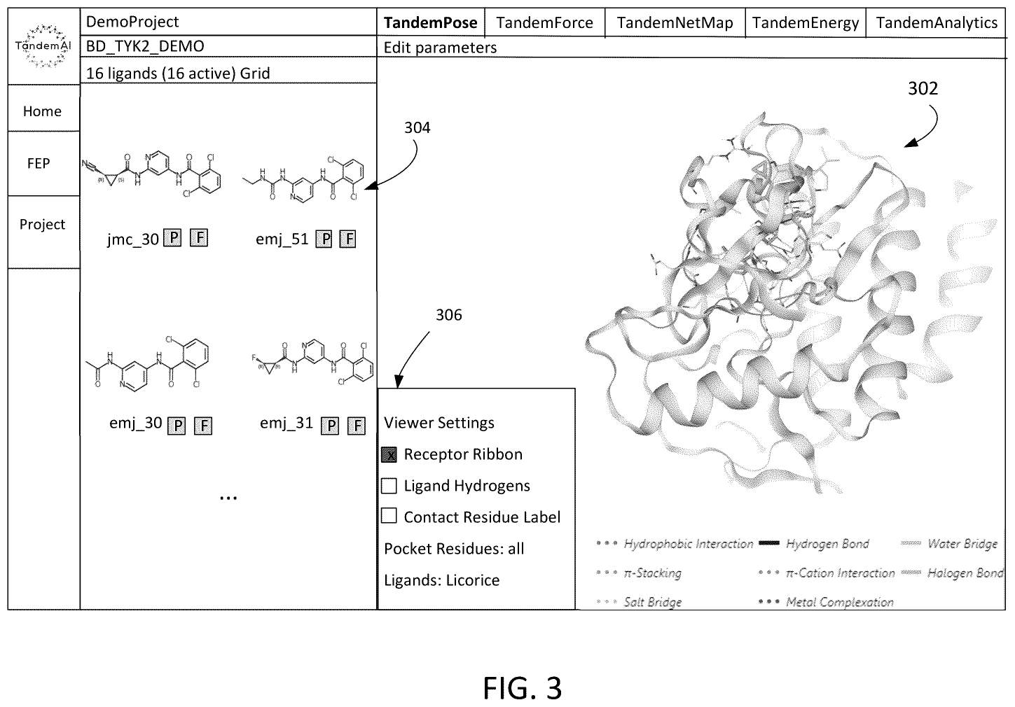

The feedback for a computational experiment may be provided separately for each task (e.g., pose, force, net mapping, energy, or analytics) associated with the computational experiment. FIG. 3 illustrates an example of feedback associated with a pose calculation that may be performed by the disclosed computational drug discovery system (e.g., as part of a computational experiment or run). As described earlier, such a pose calculation may determine the respective binding positions of a plurality of ligands with a receptor at one or more binding sites/pockets, as well as respective interactions between the ligands and the receptor (e.g., hydrogen bonds, halogen bonds, water bridges, salt bridges, hydrophobic contacts, Pi stackings, and/or Cation-Pi interactions, etc.). As shown in FIG. 3, the results of the pose calculation may be visualized through a 3D viewer 302, which may be moved, rotated, and/or zoomed in/out to examine the ligand-receptor binding from different angles and/or with different levels of details. By default, a reference ligand may be loaded with the receptor, but any other ligand in the dataset may also be added to the 3D viewer, for example, by selecting that ligand in left panel 304. And once a ligand is added to the 3D viewer, the user may control what information about the ligand is displayed in the 3D viewer. For example, a control may be provided on panel 304 for the user to toggle on and off ligand interaction visibility in the 3D viewer. Each ligand tile in panel 304 may also display basic information about the ligand (e.g., ligand name) as well as certain running results (e.g., pose (P) and/or force (F) results) for the ligand. A status icon may be provided for each ligand in the corresponding tile, as an indicator of whether the pose calculation has been completed for the ligand. In examples, the user may download the results for a ligand and/or modify a ligand (e.g., a non-reference ligand). For instance, the user may upload a new file for a ligand (e.g., to replace the original ligand pose) and that ligand file may be used in a subsequent calculation/task. The user may also upload a ligand file for a ligand whose pose calculation has failed, and the computational drug discovery system may, in response, re-determine the ligand-receptor interactions for the ligand based on the uploaded file. The user may adjust the display in 3D viewer 302 by adjusting viewer settings 306.

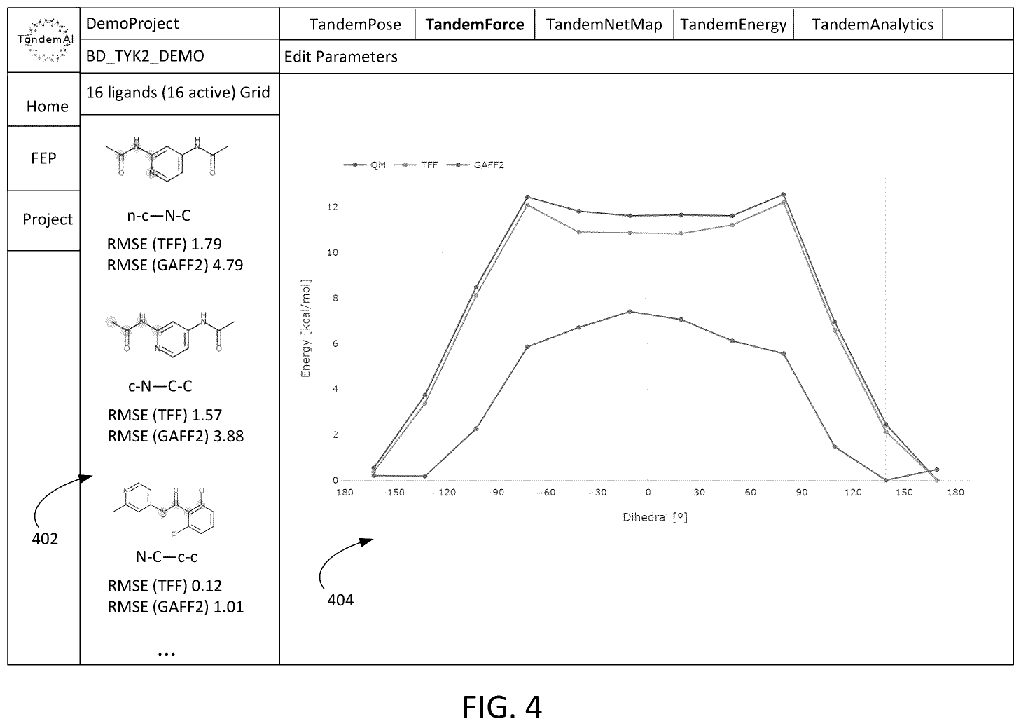

FIG. 4 illustrates an example of feedback associated with a bespoke small molecule force field parameterization calculation performed by the computational drug discovery system described herein (e.g., as part of a computational experiment). As described herein, the force field calculation may generate small molecule force fields and compare the fit between the potential energy dihedral scan as determined by a Quantum Mechanics (QM) calculation, TandemForce, and GAFF2 for each dihedral in a dataset. As shown in FIG. 4, the fragment dihedrals found in a ligand may be indicated in table 402, which may also provide a summary of all the active dihedral potential energy scans, comparing the dihedral scan potential energies determined by the TandemForce, GAFF2 and the QM calculations. The root-mean-square error (RMSE) may be reported for each comparison, with a smaller value indicating a higher level of agreement with QM predictions, and plot 404 on the right of FIG. 4 may indicate the level of agreement between the potential energy scan (PES) derived from the Tandemforce calculation and the QM PES.

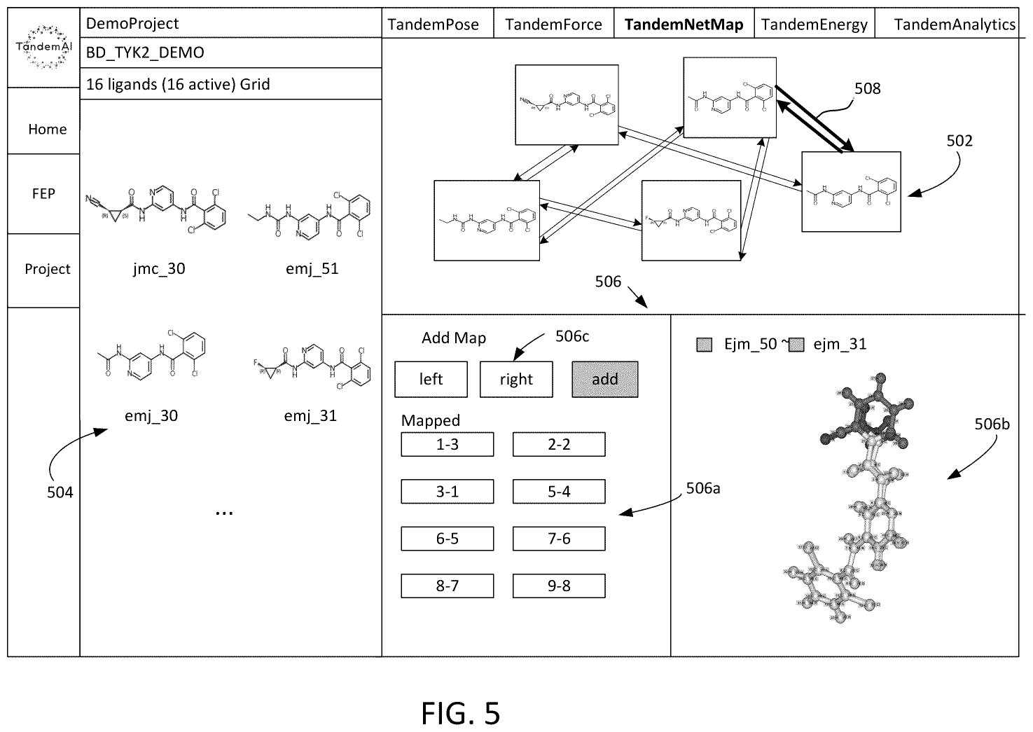

FIG. 5 illustrates an example of feedback associated with a ligands mapping (also referred to herein as network mapping or NetMap) performed by the computational drug discovery system described herein (e.g., as part of a computational experiment). The mapping (e.g., of corresponding atoms between ligands) may be generated automatically in response to detecting similar chemical and/or structural properties between ligands, or manually based on a user input that connects two or more of the ligands together (e.g., the computational drug discovery system may first present the ligands as disconnected and then allow the user to connect a pair of ligands by selecting one ligand followed by another). In either scenario, a parameter (e.g., connectivity_number) indicating the number of mappings or connections (e.g., 3, 4, etc.) from a ligand may be provided to guide the generation of the mapping network and the generated mapping may be used to perform free energy calculations (e.g., RBFE calculations) as described herein. In examples, the computational drug discovery system may be configured to display the binding free energies of one or more ligands with reference to the mapping between those ligands. For example, the computational drug discovery system may determine the relative binding free energies (RBFEs) between one or more mapped pairs of ligands and provide a representation of the determined RBFEs together with the mappings via graph 502 (e.g., a mapping network) shown in FIG. 5. Each node in the graph may represent a respective ligand while each edge (e.g., a forward edge or a reverse edge) may represent a mapping and/or an RBFE of the two ligands connected by the edge (e.g., in a forward direction or a reverse direction).

In addition to the mapping network 502, the computational drug discovery system may also display the list of ligands included in a dataset (e.g., in panel 504) and/or allow a user to examine, add, and change an edge in the mapping network 502 using the UI tools provided on panel 506. For example, in response to the user selecting a ligand from panel 504, the computational drug discovery system may highlight and/or zoom in on the ligand in mapping network 502. In response to the user selecting an edge (e.g., edge 508) from mapping network 502, the computational drug discovery system may display a list of mapped atoms from the two ligands connected by the selected edge in area 506a and/or a 3D representation of the two ligands in area 506b, where the 3D representation may indicate corresponding atoms of the two ligands that are mapped and/or atoms that are not mapped. The computational drug discovery system may further allow the user to add or edit the atom mappings between the two ligands, for example, by selecting and mapping additional atoms of the two ligands via control 506c. The computational drug discovery system may also allow the user to delete an edge or add a new edge (e.g., which may trigger a new free energy run).

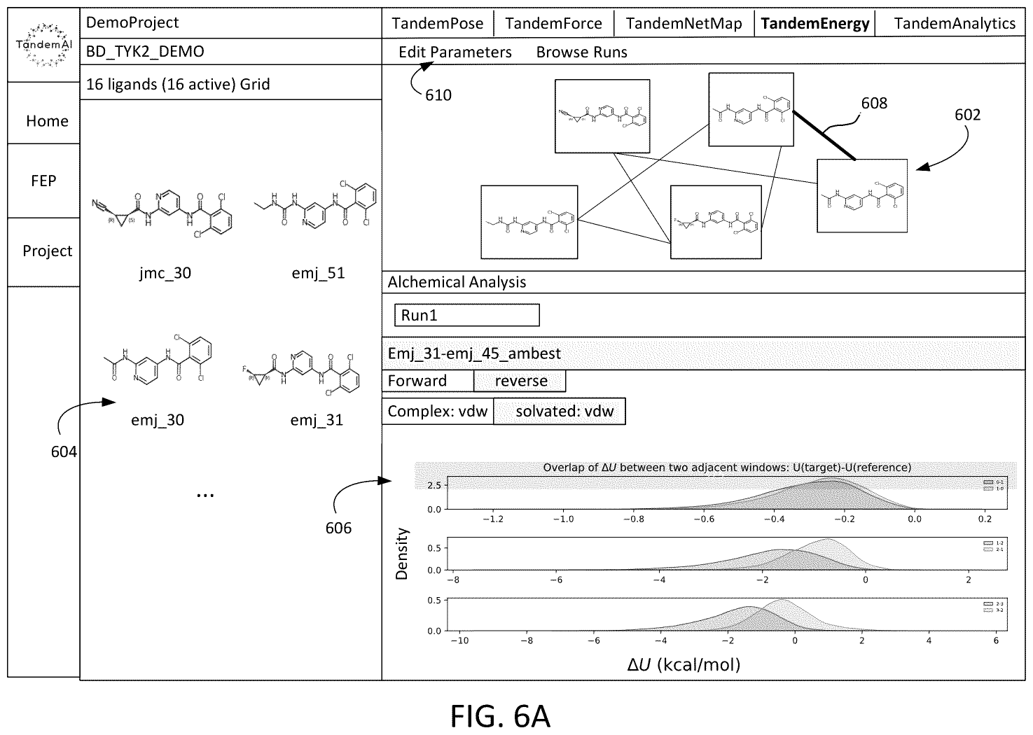

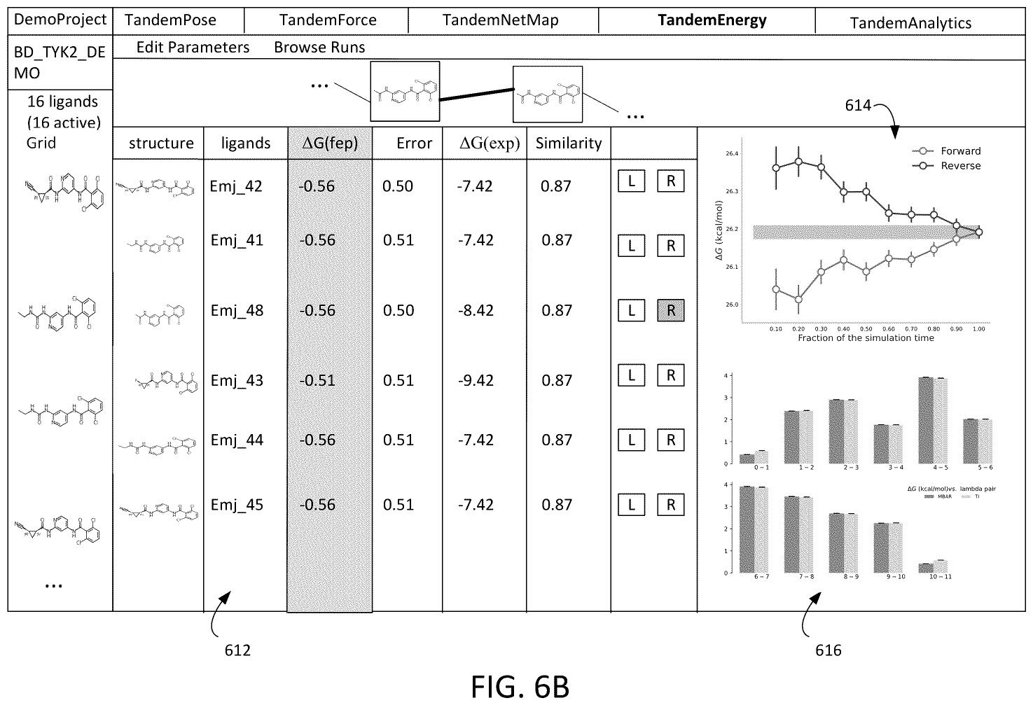

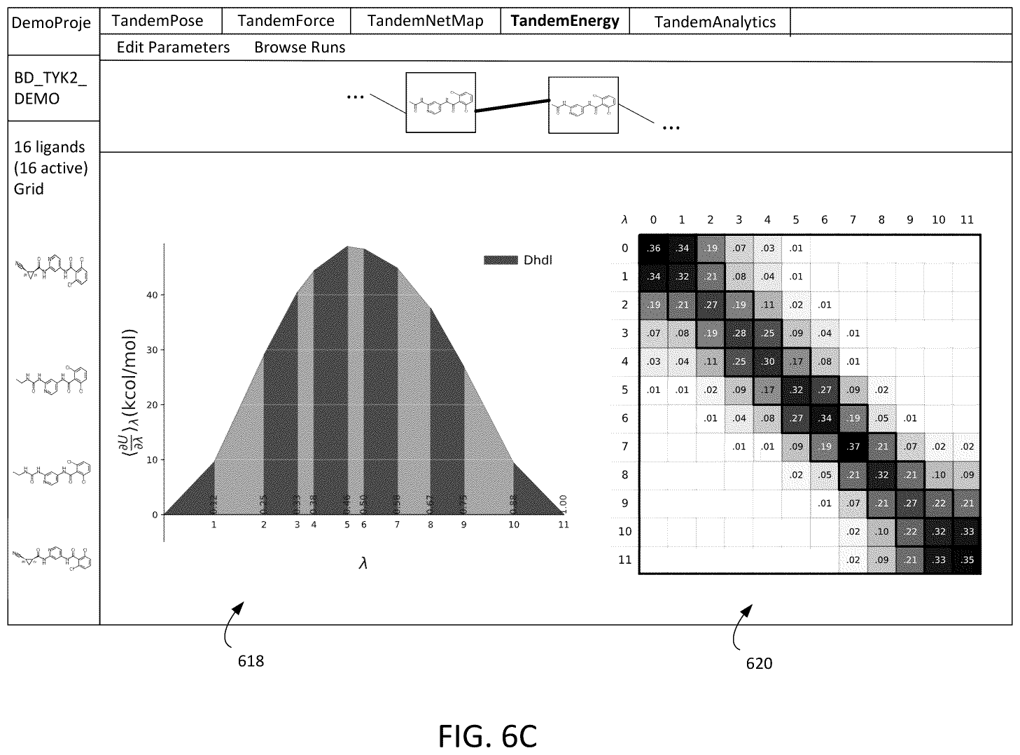

FIG. 6A, FIG. 6B and FIG. 6C illustrate examples of feedback associated with a free energy calculation performed by the computational drug discovery system (e.g., as part of a computational experiment). As shown in these figures, the free energy feedback may include the same mapping network and ligand panel (e.g., mapping network 602 and ligand panel 604 of FIG. 6A) as shown in FIG. 5, and may further include plots, charts, and/or tables summarizing various alchemical properties of the ligands in the mapping network 602. As shown in FIG. 6A, these plots, charts, and/or tables may be displayed on panel 606 in response to a user selecting an edge (e.g., edge 608) from the mapping network and the information displayed may include free energies (e.g., RBFE) and/or other alchemical properties of the ligands connected by the selected edge in a forward direction and/or a reverse direction (e.g., over time, in adjacent time windows, etc.). The user may edit the parameters associated with the free energy calculation (e.g., by clicking the “Edit Parameters” control 610) and have the calculation re-run for at least the selected edge.

As shown in FIG. 6B and FIG. 6C, the feedback from a free energy calculation may be presented in the form(s) of charts, plots, lists, tables, rankings, 3D models, etc. For example, the feedback may include a list 612 of the ligands for which the free energy calculation has been performed, which may be ranked or sorted based on various data fields or criteria including, e.g., ΔG values derived from the energy calculation, ΔG values derived from an experiment file, error rates, similarity scores (e.g., compared to a currently selected left ligand), etc. As another example, the analytics may include a plot 614 of ΔG values over a simulation time period in forward and reverse directions and a bar chart 616 of paired ΔG and lambda values, as shown in FIG. 6B. As yet another example, the analytics may include a plot 618 and a plot 620, as shown in FIG. 6C, which indicate the levels of overlap and convergence of the free energy calculations.

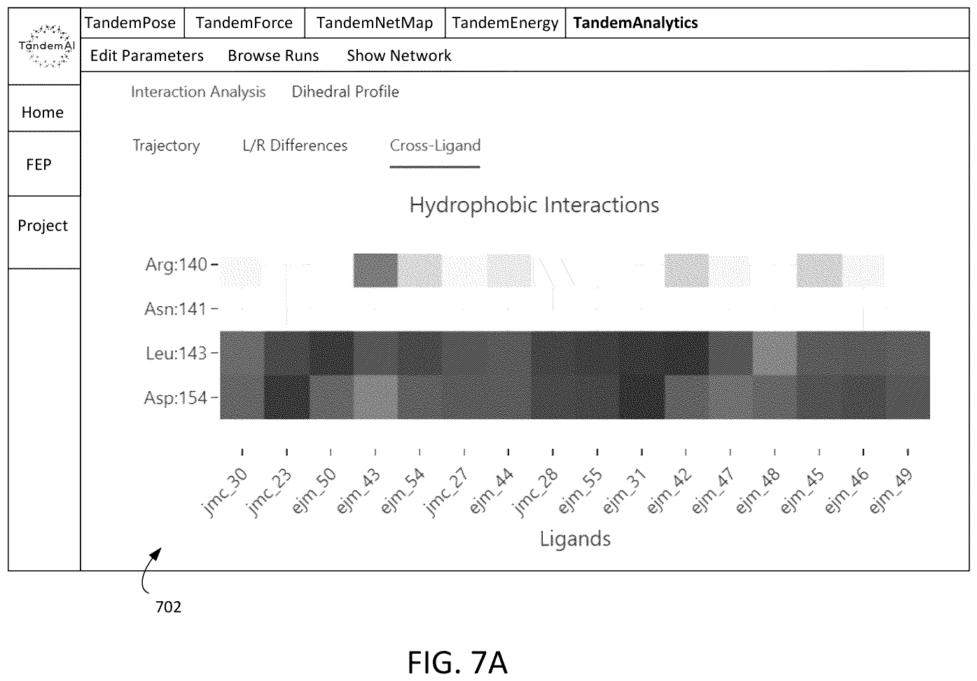

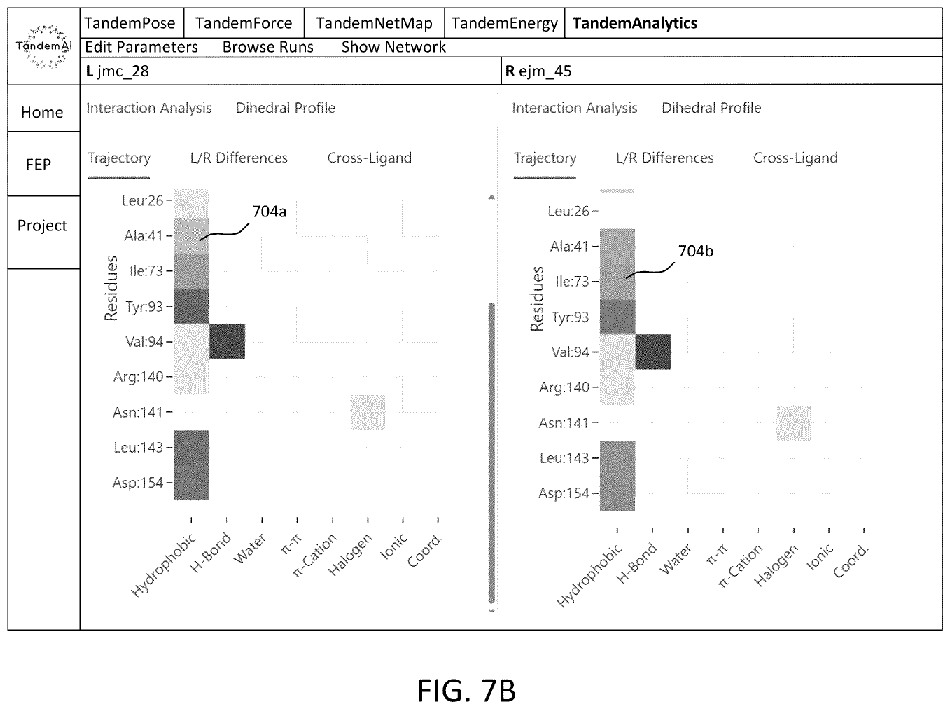

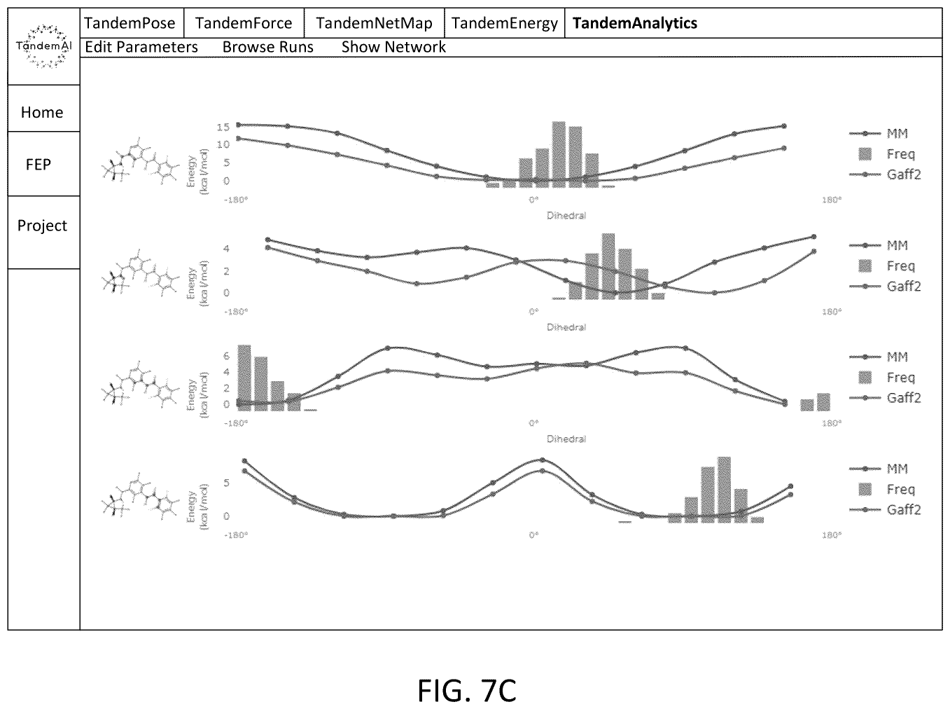

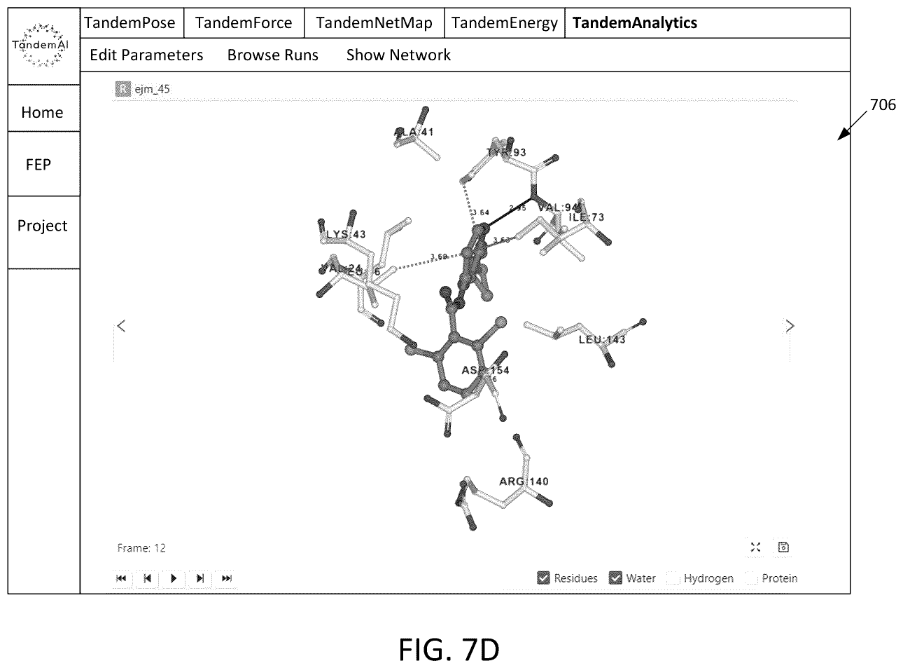

FIG. 7A, FIG. 7B and FIG. 7C illustrate examples of analytics that may be provided by the computational drug discovery system described herein. For instance, the computational drug discovery system may, as part of a computational experiment, simulate the interactions between the receptor and the plurality of ligands associated with the computational experiment and provide analytics that indicate the ligand-receptor interactions determined during the simulation. The computational drug discovery system may determine the ligand-receptor interactions based on the chemical groups on the receptor side and on the ligand side, and their respective orientations and distances at each step of the simulation. For instance, if the receptor has a positively charged group in close enough proximity to a negatively charged atom on the ligand, an electrostatic interaction may be inferred (e.g., which may be referred to as a “salt-bridge”). The computational drug discovery system may present the determined ligand-receptor interactions in various ways to allow a user of the system to inspect the molecular interaction profile of the receptor and ligands involved in the computational experiment. For example, as shown in FIG. 7A, the computational drug discovery system may provide a “Cross-Ligand” view 702 of the respective interactions between the receptor and each ligand (e.g., jmc_30, jmc_23, etc.) with respect to one or more residues (e.g., Arg: 140, Asn: 141, Leu: 143, etc.), where the different shadings may indicate the respective frequencies at which the interactions are observed during the simulation. As another example, the computational drug discovery system may allow the user to compare the pattern in which interactions occurs between the receptor and a first ligand (e.g., jmc_28) during the simulation and the pattern in which interactions occurs between the receptor and a second ligand (e.g., ejm_45) during the simulation, as illustrated by in FIG. 7B. These patterns may indicate, for example, where the interactions have occurred (e.g., with which residues), the nature of the interactions (e.g., Hydrophobic, H-bond, Water, etc.), and/or the frequencies at which the interactions are observed during the simulation (e.g., the intensity of heatmaps 704a and 704b indicates the proportion of frames an interaction is sampled in a trajectory).

The user may elect to switch to a “dihedral profile” view and the computational drug discovery system may, in response, present plots depicting the dihedral angles sampled during a trajectory associated with the selected ligands, as illustrated by FIG. 7C. The lines in these plots may show the potential energy of each dihedral, while the bars in the plots may represent the counts of frames with the dihedral in a specific angle range in all frames of a trajectory file.

As shown FIG. 7D, the computational drug discovery system may further provide a 3D viewer 706 for viewing the molecular dynamics simulation (MD) trajectory of a ligand (e.g., ejm_45). The trajectory may be presented as a MD movie comprising multiple frames, each of which may correspond to a step of the simulation and indicate the interactions between the ligand and the receptor at that step (e.g., given the corresponding orientations and distances of the ligand and the receptor). The user may use the controls shown in FIG. 7D to pause/play and/or skip through the different frames to visualize the interactions between the ligand and the receptor at different binding positions.

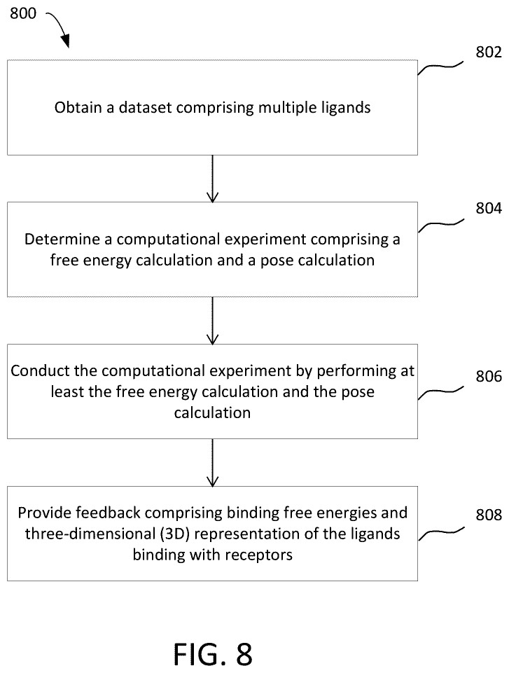

FIG. 8 illustrates an example process 800 for conducting a computational experiment in accordance with embodiments of the present disclosure. As shown in FIG. 8, the process may include obtaining a dataset at 802, where the dataset may include information regarding a plurality of ligands that a user may want to evaluate. The process may further include determining, at 804, a computational experiment to be conducted using the dataset of ligand, wherein the computational experiment may include a free energy calculation and a pose calculation. The free energy calculation may be designed to determine respective binding free energies between a receptor and the plurality of ligands in the dataset, and the pose calculation may be designed to determine respective manners in which the plurality of ligands binds with the receptor that result in the binding free energies determined by the free energy calculation. The process may further include conducting the computational experiment at 806 by performing at least the free energy calculation and the pose calculation, and providing feedback about the computational experiment at 808, wherein the feedback may include a 3D representation of the respective manners in which one or more of the plurality of ligands bind with the receptor and an indication of the respective binding free energies between the receptor and the one or more ligands.

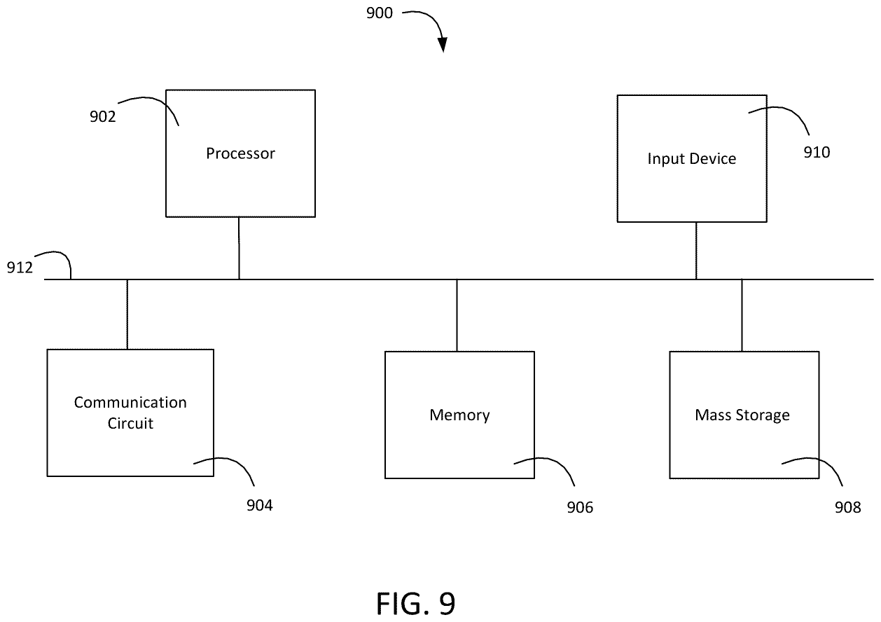

FIG. 9 illustrates an example of a computing device 900 (e.g., the first or second computing device in the drug discovery system 100 of FIG. 1) that may be configured to perform one or more of the tasks described herein. As shown, computing device 900 may include a processor (e.g., one or more processors) 902, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the tasks described herein. Computing device 900 may further include a communication circuit 904, a memory 906, a mass storage device 908, an input device 910, and/or a communication link 912 (e.g., a communication bus) over which the one or more components shown in the figure may exchange information.

Communication circuit 904 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 906 may include a storage medium (e.g., a non-transitory storage medium) configured to store machine-readable instructions that, when executed, cause processor 902 to perform one or more of the tasks described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 908 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 902. Input device 910 may include a keyboard, a mouse, a voice-controlled input device, a touch sensitive input device (e.g., a touch screen), and/or the like for receiving user inputs to computing device 900.

It should be noted that computing device 900 may operate as a standalone device or may be connected (e.g., networked, or clustered) with other devices to perform the tasks described herein. And even though only one instance of each component is shown in FIG. 9, a skilled person in the art will understand that computing device 900 may include multiple instances of one or more of the components shown in the figure.

1. A method for computational drug discovery, the method comprising:
obtaining a dataset, wherein the dataset includes information regarding a plurality of ligands;
determining a computational experiment to be conducted using the dataset, wherein the computational experiment includes a free energy calculation and a pose calculation, wherein the free energy calculation is to determine respective binding free energies between a receptor and the plurality of ligands, and wherein the pose calculation is to determine respective manners in which the plurality of ligands binds with the receptor that result in the binding free energies;
conducting the computational experiment by performing at least the free energy calculation and the pose calculation; and
providing feedback regarding the computational experiment, wherein the feedback includes a three-dimensional (3D) representation of the respective manners in which one or more of the plurality of ligands bind with the receptor, the feedback further including an indication of the respective binding free energies between the receptor and the one or more of the plurality of ligands.

2. The method of claim 1, further comprising receiving one or more user inputs that indicate the dataset and the receptor associated with the computational experiment.

3. The method of claim 1, further comprising, after initiating an original run of the computational experiment, receiving an indication of a change to the pose calculation or the free energy calculation, and initiating an additional run of the computational experiment based on the change while maintaining the original run of the computational experiment.

4. The method of claim 3, further comprising providing a graphical representation of a status of the computational experiment, wherein the graphical representation depicts the original run and the additional run of the computational experiment as two forked branches.

5. The method of claim 3, wherein the change to the pose calculation or the free energy calculation includes a change to a parameter of the pose calculation or the energy calculation.

6. The method of claim 1, wherein the free energy calculation includes a relative binding free energy (RBFE) calculation associated with at least a subset of the plurality of ligands, and wherein the feedback further includes a graphical representation of the subset of the plurality of ligands, the graphical representation including nodes and edges, each node representing a respective ligand, each edge representing a respective RBFE associated with two ligands connected by the edge.

7. The method of claim 6, further comprising, in response to receiving a user input that indicates a selection of an edge in the graphical representation, providing a 3D representation of the two ligands connected by the selected edge, wherein the 3D representation indicates corresponding atoms of the two ligands that are mapped during the RBFE calculation.

8. The method of claim 6, further comprising determining that corresponding atoms of two or more ligands have been selected and establishing a mapping between the corresponding atoms, wherein the RBFE calculation is performed based on the established mapping.

9. The method of claim 1, further comprising simulating, as part of the computational experiment, respective interactions between the receptor and each of the plurality of ligands, wherein the feedback further includes an indication of the respective interactions between the receptor and each of the plurality of ligands.

10. The method of claim 9, wherein the feedback includes a comparison of the pattern in which the interactions occur between the receptor and a first ligand, and the pattern in which the interactions occur between the receptor and a second ligand.

11. The method of claim 9, wherein the feedback includes one or more heat maps that compare the patterns in which interactions occur between the receptor and each of the plurality of ligands.

12. The method of claim 9, wherein, for each of the plurality of ligands, the feedback includes a molecular dynamics (MD) trajectory movie that depicts the interactions between the receptor and the ligand.

13. The method of claim 12, wherein the MD trajectory movie includes multiple frames, each frame corresponding to a step in the simulation and depicts the interactions between the receptor and the ligand during the step.

14. A system, comprising a first set of one or more computing devices and a second set of one or more computing devices, wherein:
the first set of one or more computing devices is configured to:
obtain a dataset that includes information regarding a plurality of ligands; and
determine a computational experiment to be conducted using the dataset, wherein the computational experiment includes a free energy calculation and a pose calculation, wherein the free energy calculation is to determine respective binding free energies between a receptor and the plurality of ligands, and wherein the pose calculation is to determine respective manners in which the plurality of ligands binds with the receptor that result in the binding free energies; and
the second set of one or more computing devices is configured to:
conduct the computational experiment by performing at least the pose calculation and the free energy calculation; and
provide feedback regarding the computational experiment, wherein the feedback includes a three-dimensional (3D) representation of the respective manners in which one or more of the plurality of ligands bind with the receptor, the feedback further including an indication of the respective binding free energies between the receptor and the one or more of the plurality of ligands.

15. The system of claim 14, wherein the first set of one or more computing devices is further configured to receive one or more user inputs that indicate the dataset and the receptor associated with the computational experiment.

16. The system of claim 14, after initiating an original run of the computational experiment, the second set of one or more computing devices is further configured to receive an indication of a change to the pose calculation or the free energy calculation, and initiating an additional run of the computational experiment based on the change while maintaining the original run of the computational experiment.

17. The system of claim 16, wherein the second set of one or more computing devices is further configured to provide a representation of the status of the computational experiment that depicts the original run and the additional run of the computational experiment as two forked branches.

18. The system of claim 14, wherein the free energy calculation includes a relative binding free energy (RBFE) calculation associated with at least a subset of the plurality of ligands, and wherein the feedback provided by the second set of one or more computing devices further includes a graphical representation of the subset of the plurality of ligands, the graphical representation including nodes and edges, each node representing a respective ligand, each edge representing a respective RBFE associated with two ligands connected by the edge.

19. The system of claim 18, wherein the second set of one or more computing devices is further configured to determine that corresponding atoms of two or more ligands have been selected, establish a mapping between the corresponding atoms, and perform the RBFE calculation based on the established mapping.

20. The system of claim 14, wherein the second set of one or more computing devices is further configured to simulate, as part of the computational experiment, respective interactions between the receptor and each of the plurality of ligands, and wherein the feedback further includes an indication of the respective interactions between the receptor and each of the plurality of ligands.
